(19) United States
(12) Patent Application Publication
(10) Pub. No.: US 2004/0214884 A1
(43) Pub. Date: Oct. 28, 2004

(54) Method for supplementing magnesium in an animal

(76) Inventors: Brent P. Hagen, Brigham City, UT (US); Wayne E. White, Thousand Oaks, CA (US); Shane R. Shupe, Harrisville, UT (US)

Correspondence Address: Thompson E. Fehr, Suite 300, Goldenwest Corporate Center, 5025 Adams Avenue, Ogden, UT, 84403, US

(21) Appl. No.: 10/425,160

(22) Filed: Apr. 28, 2003

(51) Int. Cl. A61K 31/325

(52) U.S. Cl. 514/476

(57) ABSTRACT

A method for supplementing magnesium in an animal by administering a stable complex of magnesium and aspartic acid having the formula MgCOOCH₂CIINII₂COO, which is highly soluble in water, to the animal. One form of the commercial product of the compound has the magnesium monoaspartate dissolved in water. Another form has the magnesium monoaspartate added to a food.

### BACKGROUND OF THE INVENTION

1. Field of the Invention

This invention relates to the use of magnesium monoaspartate for incorporating magnesium as a dietary supplement within an animal, including a human being.

2. Description of the Related Art

Sixteen patents are known to disclose magnesium amino acid chelates or magnesium aspartate. These are U.S. Pat. Nos. 4,738,856; 5,143,732; 5,284,674; 5,393,532; 5,460,944; 5,504,055; 5,516,925; 5,824,353; 5,858,403; 5,895,652; 5,939,076; 6,051,236; 6,159,530; 6,197,815; 6,207,204; and 6,248,368. Such compounds are, however, distinct from that of the present invention. In particular, those that disclose magnesium aspartate do not disclose magnesium monoaspartate. And although U.S. Pat. No. 4,855,289 discloses magnesium monoaspartate hydrochloride, it does not disclose magnesium monoaspartate, itself.

U.S. Pat. No. 5,006,516 does disclose magnesium monoaspartate, but only as one ingredient in an agent “for the prophylaxis of cardiac infarctions and the prevent of reinfarctions.” The disclosure indicates that the magnesium monoaspartate “ . . . can be produced by dissolving 225 g magnesium-L-diaspartate X4H₂O in 250 ml distilled water, and then slowly adding a potassium hydroxide solution (80 g KOH tablet 85% in 100 ml distilled water) in the form of drops until a pH value of 10.2-10.6 is reached. The clear solution is stirred for one hour. The volume of the solution is then reduced to between 250-275 ml at 40° C. in a water jet vacuum (approximately 14 torr) and is left standing at room temperature. The solution crystallizes after about four days. The crystals are removed in a water jet vacuum by means of a frit and then washed, first with a small quantity of distilled water and then with acetone. The crystals are then dried at about 80-90° C. until the acetone odor disappears.”

### BRIEF SUMMARY OF THE INVENTION

The compound that is the subject of the present invention is a stable complex of magnesium and aspartic acid, which has been determined to be highly soluble in water and, in fact, in both neutral and acid solutions. Such solubility combined with a relatively high content of magnesium makes the compound beneficial as a dietary supplement and for use in fortified foods and beverages where a concentrated, soluble magnesium source is desired.

The magnesium monoaspartate is, thus, preferably administered to the animal after having been dissolved in water or after, using any technique that is well known in the art, having been added to a food.

### DETAILED DESCRIPTION OF THE INVENTION

The compound of the present invention is, as indicated above, magnesium monoaspartate.

The formula for this compound is MgCOOCH₂CHNH₂COO.

Since it is highly soluble in water, it is, as mentioned above, beneficial as a dietary supplement. Also, it is convenient, but not necessary, to dissolve the compound in water in order to create a commercial form of the compound. Additionally, as indicated above, the magnesium monoaspartate may, using any technique that is well known in the art, be added to a food.

An example of a method for manufacturing the magnesium monoaspartate is as follows:

### EXAMPLE

Magnesium oxide was suspended in water to make a 20 percent, by weight, magnesium oxide slurry. An equimolar quantity of 1-aspartic acid was added and reacted with the magnesium oxide in the slurry. The product was oven dried.

The resultant magnesium monoaspartate is administered to an animal independently or, preferably, dissolved in water or combined with a food.

We claim:

1. A method for supplementing magnesium in an animal, which comprises:
administering to an animal a composition of matter having the formula MgCOOCH₂CHNH₂COO.

2. The method for supplementing magnesium in an animal as recited in claim 1, wherein:
the administering comprises:
dissolving the MgCOOCH₂CHNH₂COO in water; and
providing the water containing the MgCOOCH₂CHNH₂COO to the animal.

3. The method for supplementing magnesium in an animal as recited in claim 1, wherein:
the administering comprises:
combining the MgCOOCH₂CHNH₂COO with a food; and
providing the food containing the MgCOOCH₂CHNH₂COO to the animal.

4. A method for supplementing magnesium in an animal, which comprises:
making MgCOOCH₂CHNH₂COO by a process, which comprises:
suspending a sufficient quantity of magnesium oxide in water to create a slurry that is twenty percent magnesium oxide by weight;
adding an equimolar quantity of 1-aspartic acid to react with the magnesium oxide in the slurry; and
drying a composition constituting MgCOOCH₂CHNH₂COO resulting from said suspending and said adding; and
administering to an animal the MgCOOCH₂CHNH₂COO made by said process.

5. The method for supplementing magnesium in an animal as recited in claim 4, wherein:
the administering comprises:
dissolving the MgCOOCH₂CHNH₂COO in water; and
providing the water containing the MgCOOCH₂CHNH₂COO to the animal.

6. The method for supplementing magnesium in an animal as recited in claim 4, wherein:
the administering comprises:
combining the MgCOOCH₂CHNH₂COO with a food; and
providing the food containing the MgCOOCH₂CHNH₂COO to the animal.
